(19) United States
(12) Patent Application Publication
(10) Pub. No.: US 2018/0318115 A1
(43) Pub. Date: Nov. 8, 2018

(54) FRACTURE RESISTANT STENT

(71) Applicant: Veniti, Inc., St. Louis, MO (US)

(72) Inventors: Abha Chinubhai, Fremont, CA (US); Abha CHINUBHAI, Fremont, CA (US)

(21) Appl. No.: 15/911,002

(22) Filed: Mar. 2, 2018

Related U.S. Application Data
(60) Provisional application No. 62/466,641, filed on Mar. 3, 2017.
(60) Provisional application No. 62/466,728, filed on Mar. 3, 2017.

(51) Int. Cl. A61F 2/91

(52) U.S. Cl. CPC A61F 2/91; A61F 2250/0098; A61F 2210/0014; A61F 2230/006; A61F 2230/0013; A61F 2230/0054; A61F 2230/0091; A61F 2250/0067

(57) ABSTRACT

A stent includes a first stent portion formed from a first stent type and a second stent portion formed from a second stent type. The second stent portion is coupled to the first stent portion, and the first stent type is different than the second stent type. The stent portions can be braided. The stent can taper from a first end to a second end.

### CROSS-REFERENCE TO RELATED APPLICATIONS

This nonprovisional application claims the benefit of and priority to U.S. Provisional Application No. 62/466,641, filed Mar. 3, 2017 and U.S. Provisional Application No. 62/466,728 filed Mar. 3, 2017. The disclosures of the prior applications are hereby incorporated by reference herein in their entirety.

### BACKGROUND

Medical devices, such as endoluminal prostheses, including stents, or other implantable structures can be used in different procedures. For example, the prostheses may be placed in the arterial system, the venous system, or any other portion of the body. Stents may also be used to deliver drugs to tissue, support tissue, or maintain patency of body lumens, as well as perform other functions.

Stents are typically delivered via a catheter in an unexpanded configuration to a desired location in the body. The combined stent and catheter is typically referred to as the stent delivery system. Once at a desired location, the stent is expanded and implanted into the body lumen. Examples of locations in the body include, but are not limited to, arteries (e.g. aorta, coronary, carotid, cranial, iliac, femoral, etc.), veins (e.g. vena cava, jugular, iliac, femoral, hepatic, subclavian, brachiocephalic, azygous, cranial, etc.), as well as other locations including the esophagus, biliary duct, trachea, bronchials, duodenum, colon, and ureter.

Typically, a stent will have an unexpanded configuration with reduced diameter for delivery and an expanded configuration with expanded diameter after placement in the vessel, duct, or tract. Some stents are self-expanding, and some stents are mechanically expanded with a radial outward force applied from within the stent (e.g., with a balloon). Some stents have one or more characteristics common to both self-expanding and mechanically expandable stents.

In many stent delivery systems, particularly those used to deliver a self-expanding stent, the stent is typically retained on the catheter in its unexpanded form with a constraining member or other retention device such as a sheath or outer shaft. The stent may be deployed by retracting the outer shaft from over the stent. To prevent the stent from being drawn longitudinally with the retracting shaft, many delivery systems provide the catheter shaft with a pusher, bumper, hub, holder or other stopping element.

Precise delivery of stents can be challenging. In the case of balloon expandable stents, the stent may foreshorten as the stent radially expands. As a result, the change in length must be taken into account when deploying the stent at the treatment site. In the case of self-expanding stents, due to the elastic nature of the stents, these stents may “jump” away from the delivery catheter during deployment. Additionally, depending on the anatomy being treated, this may add further challenges to accurate stent delivery.

In certain parts of the anatomy, longer stents may be needed to treat longer lesions or treatment regions. For example, with ilio-femoral and ilio-caval stenting, much longer stents are often used as compared with stenting of coronary lesions. This type of venous stenting may be used for the treatment of nonthrombotic iliac vein lesions (NIVL) and post-thrombotic syndrome (PTS), whereby the profunda and the inferior vena cava can be partially or completely blocked (or “stent jailed”) by the stent if the stent is not placed accurately after deployment. Because the stents are longer, known stents are often more difficult to load onto a delivery catheter, and may buckle during the loading process when a radial force is applied to the stent to reduce the stent's diameter.

Additionally, deployment forces of radially strong or large diameter self-expanding stents can be relatively high. Furthermore, deployment forces can be equally high with stents that are longer in length due to the added friction between the stent and a constraining or protective sheath. These high deployment forces may also cause the stent to axially or radially buckle when loaded or deployed because the longer stents are less supported and less rigid, resulting in buckling during deployment. This is of particular concern when long self-expanding stents are used.

Moreover, the risk of placing stents in the venous system across the inguinal ligament may lead to fracture of the stent due to the flexure of the region near the inguinal ligament. Similarly, arterial stenting across joints has an increased risk of in-stent focal neointimal hyperplasia and compression or fracture of the stent by joint motion with decreased long-term patency.

### SUMMARY

In an embodiment, a stent includes at least one first stent portion formed from a first stent type and at least one second stent portion formed from a second stent type. The second stent portion is coupled to the first stent portion, and the first stent type is different than the second stent type.

In another embodiment, a stent includes a plurality of filaments forming a tubular structure, a first end of the tubular structure having a first property, and a second end of the tubular structure having a second property. The first property is different than the second property.

In another embodiment, a stent includes a plurality of rings, each of the rings having a plurality of struts. The stent further includes a plurality of bridges interconnecting at least some of the rings, the plurality of bridges comprising a plurality of braces, wherein the plurality of braces has a length greater than the length of each of the rings.

### BRIEF DESCRIPTION OF THE DRAWINGS

FIG. 1 illustrates a braided stent according to an embodiment.

FIG. 2 illustrates a braided stent with folded ends according to an embodiment.

FIG. 3 illustrates a braid supported by end stents according to an embodiment.

FIG. 4 illustrates a hybrid stent according to an embodiment.

FIG. 5 illustrates a singular filament stent according to an embodiment.

FIG. 6 illustrates a stent covered in a polymeric graft according to an embodiment.

FIG. 7 illustrates a tapered stent according to an embodiment.

FIG. 8 illustrates a bifurcated stent according to an embodiment.

FIG. 9 illustrates an extra-long braced stent in a radially collapsed configuration according to an embodiment.

FIG. 10 illustrates a helical braced stent according to an embodiment.

### DETAILED DESCRIPTION OF EMBODIMENTS

The following detailed description of certain embodiments will be better understood when read in conjunction with the appended drawings. To the extent that the figures illustrate diagrams of the various portions of various embodiments, the various portions are not necessarily indicative of the division between various components. Thus, for example, one or more of the portions may be implemented in a single or multiple piece design.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments provide a stent that has increased fracture resistance, as well as improved deployment. For example, superelastic filaments are used in various embodiments for stenting in the venous system. The stents in some examples have a braid or weave design. In some embodiments, the filaments can be formed from a material with shape memory or that are polymeric in nature. This creates a flexible stent with sufficient chronic outward force and radial resistive force to maintain lumen patency and accommodate external compression, anatomical bending and tortuosity.

Various embodiments, thus, provide a stent that avoids or has reduced potential for buckling during delivery, allows the stent to overcome the excessive friction and avoid the bind up of the device during stent release and has increase fracture resistance once deployed. Thus, incomplete or incorrect release of the stent is reduced, as well as increasing the life of a deployed stent.

The stent in some examples is a self-expanding stent made from a material that is resiliently biased to return to a pre-set shape. These materials can include, for example, superelastic and shape memory materials that can expand to an implanted configuration upon delivery or through a change in temperature. The self-expanding stents can be formed from a wide variety of materials including, but not limited to, nitinol (a nickel titanium alloy), spring steel, shape-memory polymers, etc. In various embodiments the stents can be formed from any shaped memory metal.

The various embodiments can be implemented in a variety of different medical devices, including endoluminal prostheses such as stents, or other implantable structures. The prostheses can be placed in the arterial system, the venous system, or any other portion of the body. The stents can also be used to deliver drugs to tissue (e.g., by coating the filaments of the stent), support tissue, or maintain patency of body lumens, as well as performing other functions.

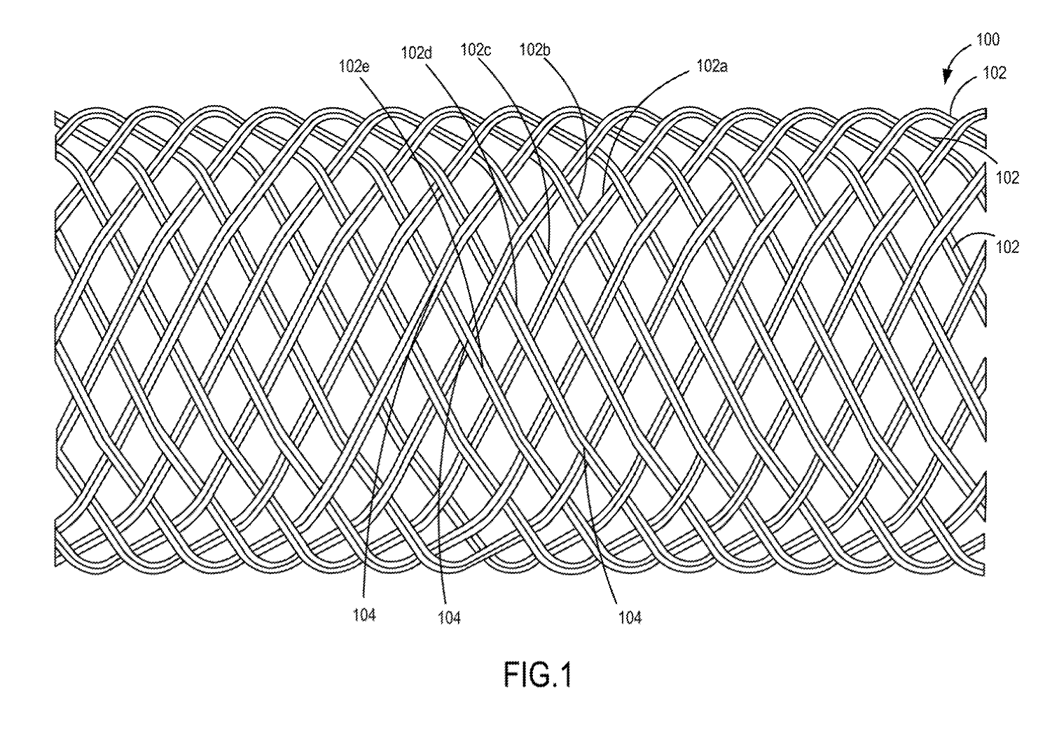

More particularly, some embodiments include a stent design having a plurality of filaments that are braided or woven/knitted to create a flexible stent that is resistant to fracture of the stent. These braids can be constrained at various overlapping. Specifically, as shown in FIG. 1, a stent 100 includes filaments 102 that are interwoven to form a tubular stent (e.g., forming a lumen therein). The filaments 102 in various embodiments are multi-strand elements (e.g., formed from twisting or otherwise combining multiple stands). The braid pattern of the filaments 102 can be varied to increase the radial strength by increasing the braid angle and number of filaments 102 in that region (e.g., along a portion of the stent 100). It should be noted that the overlapping configuration of the filaments 102 also me be varied as desired or needed. For example, although the stent 100 illustrates a repeating pattern of the overlapping shown as one filament 102a overlapping three filaments 102b, 102c and 102d, and then being overlapped by one filament 102e, which pattern is repeated circumferentially along the stent 100, the pattern can be varied to have different numbers of overlaps, as well as a non-repeating pattern. For example, alternating filaments 102 can overlap each other in some embodiments. It should also be noted that different numbers of wires can be woven together to form the braided arrangement (e.g., sixteen wires or thirty-two wires). It should further be noted that various embodiments can include different configurations, such as multiple filaments that form a single wire, a single solid wire, a strip, etc.

Thus, the structure of the stent 100 reduces foreshortening by joining single or multiple longitudinal axial fibers defining the filaments 102 (e.g. stitched, welded, mechanically constrained using metal or polymer) selectively along the entire length of the stent 100 at joining points 104. The stent 100 and the polymeric filaments 102 can also be used to deliver drugs to tissue, support tissue, or maintain patency of body lumens in some embodiments. For example, antiplatelet agents, anticoagulant agents, antimicrobial agents, anti-inflammatory agents, and other drugs can be supplied in the stent 100 to reduce the incidence of restenosis or to maintain lumen patency.

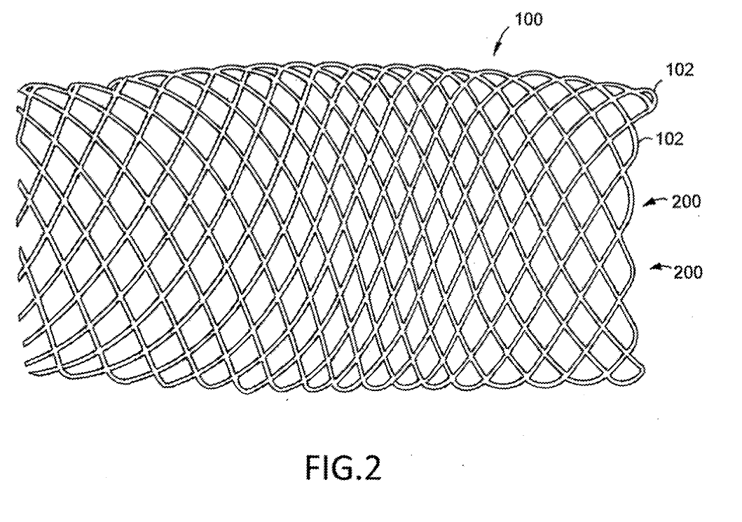

Variations and modifications are contemplated by the present disclosure. For example, as illustrated in FIG. 2, the stent 100 having the braided configuration includes folded ends 200. More particularly, in this embodiment, the ends of the filaments 102 are folded back to prevent sharp edges at the end of the stent (e.g., form a smooth end). As another example, the filaments 102 can be me made radiopaque. In one embodiment, single or multiple radiopaque filaments 102 (e.g., a Pt/W wire) can be included as part of the braid structure to provide radiopacity to the entire stent 100. Thus, weaving a single or multiple radiopaque filaments 102 into the braid pattern provides radiopacity. It should be noted that the number and arrangement of the radiopaque filaments 102 can be varied as desired or needed. In some embodiments, the stent is formed from solid wires.

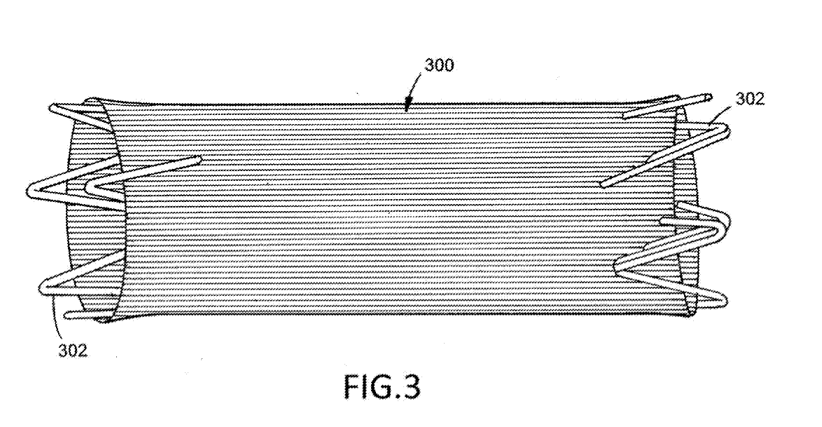

In another embodiment, as shown in FIG. 3, a braid 300 is supported by two end stents 302 that are more rigid. In one example, the end stents 302 are configured as ring stents that provide radial resistance and help prevent migration, whereas the braid 300 supports the lumen and maintains patency. The end stents 302 can be different types of stents, such as laser cut or wireform stents. The end stents 302 are joined to the braid 300 (or weave), for example, by stitching, welding or mechanical, as appropriate.

Thus, in this embodiment, a combination stent, braid and laser cut or braid and wireform stent is provided where both or either can be made using shaped memory material. The braid 300 can be polymeric or metallic. The end stents 302 and the polymeric filaments of the braid 300 can be used in some examples to deliver drugs to tissue, support tissue, or maintain patency of body lumens. Antiplatelet agents, anticoagulant agents, antimicrobial agents, anti-inflammatory agents, and other drugs can be supplied to reduce the incidence of restenosis or to maintain lumen patency. It should be noted that the braid 300 can be formed from different materials. For example, in one embodiment, the braid 300 is formed from a nickel titanium (nitinol) mesh.

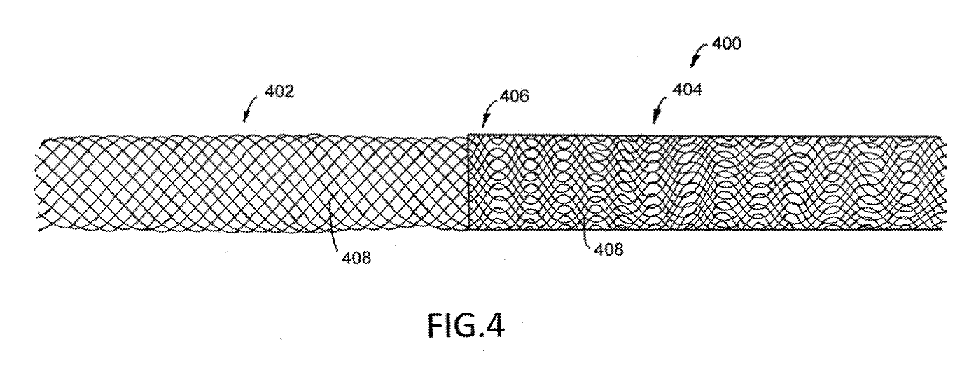

Another embodiment of a stent 400 is illustrated in FIG. 4 and configured as a hybrid stent. In particular, in the illustrated embodiment, a braided stent 402 (braided stent portion defining a first stent type) is joined to a laser cut or wireform stent 404 (laser cut or wireform stent portion defining a second stent type) at a join region 406. It should be noted that the braided stent 402 may be embodied, for example, as the stent 100 (illustrated in FIG. 1). It should also be noted that the braided stent 402 and the laser cut or wireform stent 404 can be coupled at the join region 406 using any suitable joining technique. In various embodiments, the hybrid design prevents migration, elongating (relieving radial forces) and/or luminal loss and/or crushing thereof. Moreover, the hybrid design reduces the risk of fracture when placing stents in the venous system across the inguinal ligament that may otherwise result due to the flexure of the region near the inguinal ligament. Various embodiments reduce different types of fractures results from stress and alternating stress from bending, axial movement or compression and/or radial expansion or movement. The different portions of the stent 400, namely the braided stent 402 and the laser cut or wireform stent 404 can be placed relative to the inguinal ligament to reduce the risk of fracture (e.g., braided stent 402 at bend region of inguinal ligament).

Although the stent 400 is illustrated as having generally equal length portions of the braided stent 402 and the laser cut or wire form stent 404, the length and/or diameter of each, or portions thereof, may be varied as desired or needed, such as based on design characteristics for the stent 400. Additionally, in some embodiments, multiple braided stent 402 and laser cut or wireform stent 404 portions can be joined together. For example, a braided stent 402 can be joined to both ends of the laser cut or wire form stent 404 instead of just one end as illustrated in FIG. 4.

The configuration of the stent 400 combines the flexibility of a braided stent and the radial resistive force of a laser cut stent that helps prevents stent migration. Thus, the stent 400 defines a combination stent, braid and laser cut or braid and wireform stent where both or either can be made using shaped memory material. The stent 400 and in particular, the polymeric filaments 408, can be used to deliver drugs to tissue, support tissue, or maintain patency of body lumens. Antiplatelet agents, anticoagulant agents, antimicrobial agents, anti-inflammatory agents, and other drugs can be supplied in stents to reduce the incidence of restenosis or to maintain lumen patency. It should be noted that the braided stent 402 and the laser cut or wireform stent 404 can be any type or configuration of stent. In one example, the laser cut or wireform stent 404 can be provided as described in U.S. Pat. No. 9,233,014.

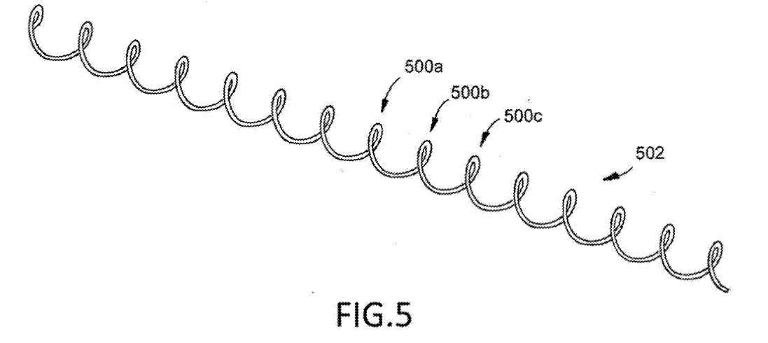

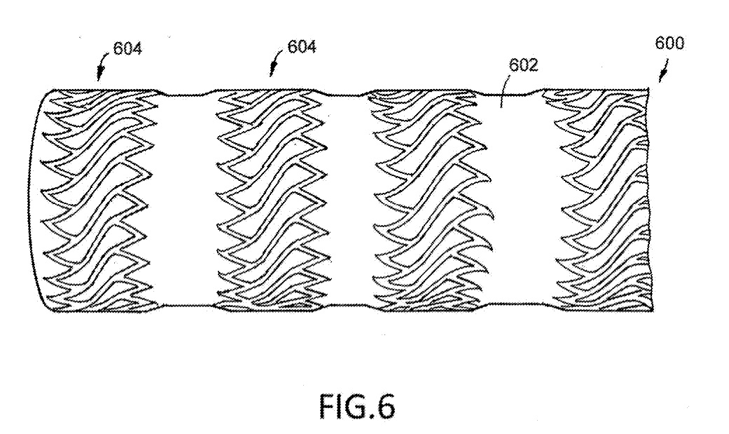

In another embodiment, as illustrated in FIG. 5, a singular filament 500 is used to form a coil 502. The coil 502, in one example, is held axially by longitudinal filaments to provide axial rigidity and assist with deliverability of the stent. For example, non-consecutive filaments 500a, 500b, and 500c defining the longitudinal filaments can be used to join each consecutive coil. Thus, in this embodiment, the coiled stent defined by the coil 502 gets additional axial rigidity by longitudinal filaments that connect each coil to the adjacent coil or selectively along the length. The longitudinal filament can be radiopaque, metallic or polymeric in nature. The stent defined by the coil 502 and in particular, the polymeric filaments 500, can be used to deliver drugs to tissue, support tissue, or maintain patency of body lumens. Antiplatelet agents, anticoagulant agents, antimicrobial agents, anti-inflammatory agents, and other drugs can be supplied in stents to reduce the incidence of restenosis or to maintain lumen patency.

It should be noted that different portions of the coil 502 formed from different coil elements, such as different longitudinal filaments, can be formed from different materials. For example, one or more coil portions can be formed from a different material than one or more other coil portions and joined together.

In another embodiment, a stent 600 is covered by a non-dense polymeric graft 602. More particularly, the stent 600 is formed of singular rings 604 (e.g., wireform or laser cut stent rings) that are connected solely by the graft 602 or connected by fewer filaments making the connection portion more flexible and less metal dense. Thus, the stent 600 is configured having a polymer tube with distinct rings 604 imparting spaced apart or spaced at variable distances. Additionally, the rings 604 can have the same width or different widths. The rings 604 can be, for example, laser cut, wireform or braided. The stent 600 and in particular, the polymeric filaments, can be used to deliver drugs to tissue, support tissue, or maintain patency of body lumens. Antiplatelet agents, anticoagulant agents, antimicrobial agents, anti-inflammatory agents, and other drugs can be supplied in stents to reduce the incidence of restenosis or to maintain lumen patency. In some examples, the polymeric tube can have slots cut out and/or fenestrations in a pattern to create channels and/or openings for blood flow, thereby preventing jailing off for a vessel. The polymeric slots can be made radiopaque for ease of navigation and placement.

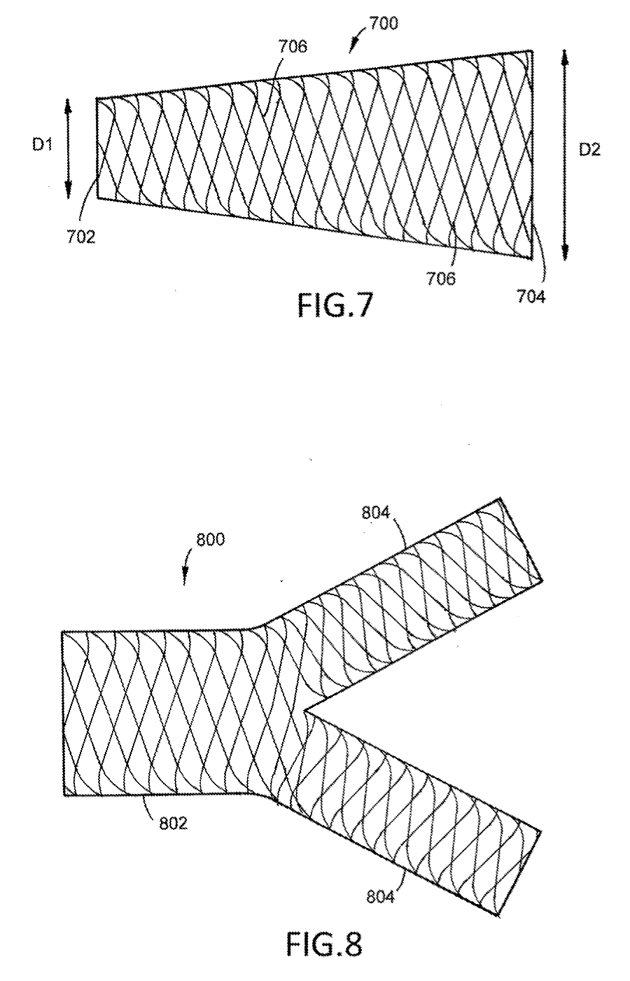

In another embodiment, a stent 700 as shown in FIG. 7 is configured as tapered stent that has a changing diameter along an axial length thereof. For example, the stent 700 has a first diameter (D1) at one end 702 and a different second diameter (D2) at a second end 704. In the illustrated embodiment, D2 is greater than D1. It should be appreciated that the amount of change in diameter between the first and second ends 702 and 704, as well as the degree or slope of the change can be varied. For example, the change in diameter from the first send 702 to the second end 704 can be a linear change in some embodiments, but can be a non-linear change in other embodiments. Additionally, the filaments 706 forming the stent 700, although shown in a braided design, can take different designs and patterns. That is, the stent 700 can be any type or configuration of stent, such as the braided stent 402 or the laser cut or wire form stent 404 as illustrated in FIG. 4.

In another embodiment, as illustrated in FIG. 8, a stent 800 is configured in a bifurcated stent design having a main stent portion 802 and bifurcated stent portions 804 that split off from the main stent portion 802. The length and diameter of the different portions can be varied as desired or needed. In one embodiment, the bifurcated stent portions 804 are leg portions configured to be placed into the left and right iliac. Using the stent 800, two stents like a double barrel do not have to be placed into the IVC.

In one embodiment, the main stent portion 802 defines a large stent in the IVC that has bifurcated stent portion 804 placed into the iliac (e.g., left iliac) and a separate stent that defines the other bifurcated stent portion 804 that connects to this from the other iliac (e.g., right iliac). Thus, in one embodiment, a joined arrangement is provided. However, other configurations are contemplated, such as having different splits in the various portions.

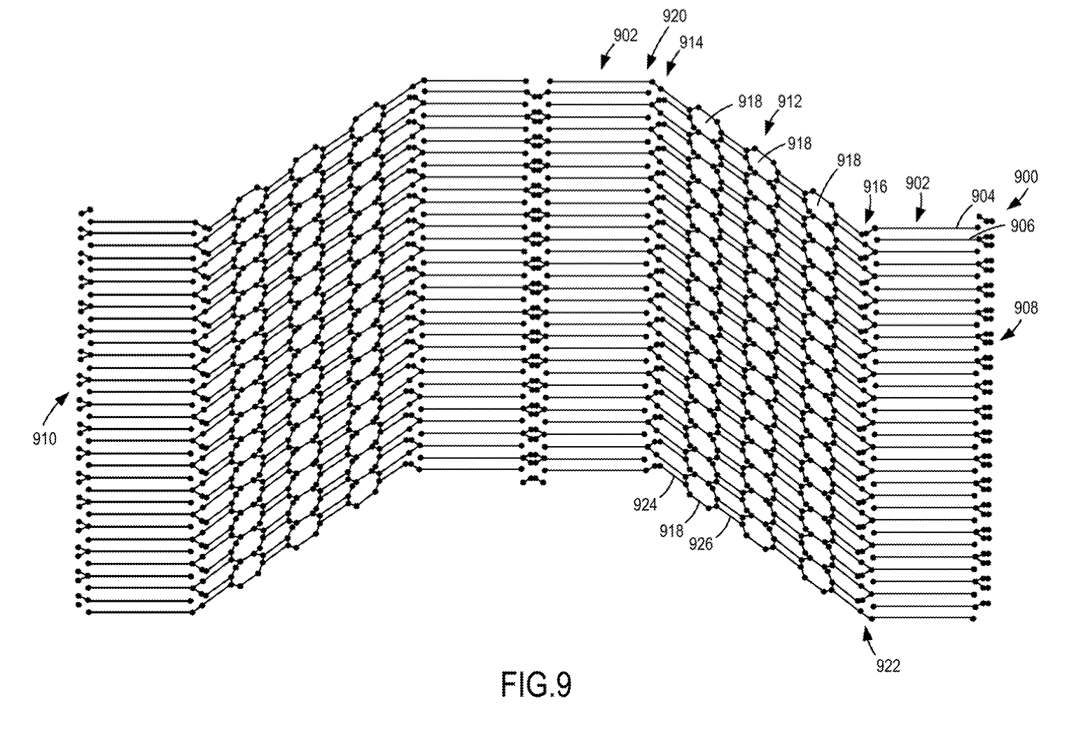

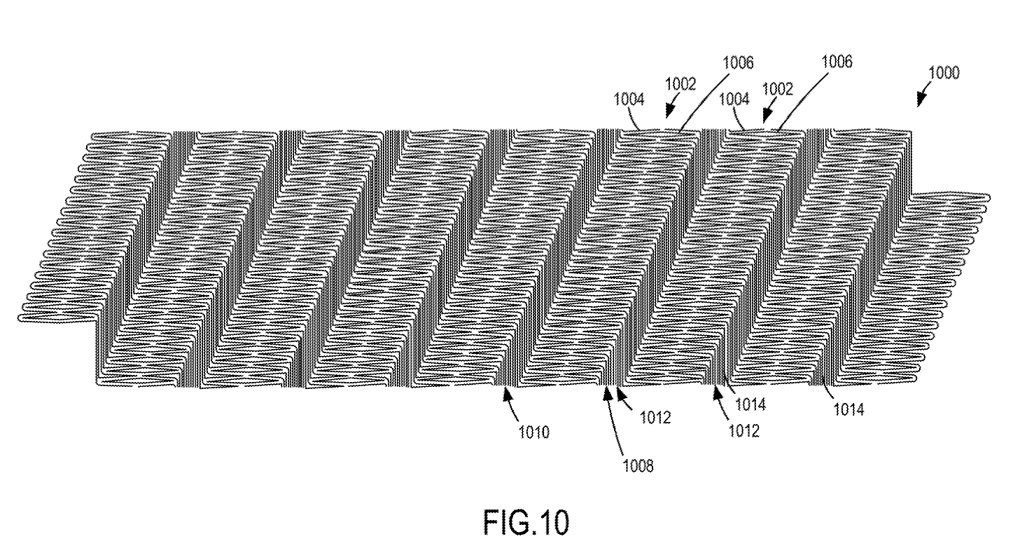

Some embodiments provide a stent used for treating longer lesions or longer treatment regions that has greater structural support and rigidity in order to resist buckling or unwanted deformation during loading onto a delivery system or during deployment in a patient. More particularly, and as illustrated in FIG. 9, a stent 900 includes a plurality of radially expandable rings 902 each having a contracted configuration suitable for delivery and a radially expanded configuration for engaging and supporting tissue. Each ring 902 is formed from a plurality of interconnected struts 904, with adjacent struts 904 in each ring 902 being connected together with a connector 906, and each ring having a proximal end 908, and a distal end 900. The plurality of rings 902 are coaxially aligned with one another to form a longitudinal axis. The distal end 910 of one ring 902 faces the proximal end 908 of the adjacent ring 902.

The stent 902 also has a plurality of extra-long bridges 912 disposed between adjacent rings 902. The plurality of the bridges 912 couple adjacent rings 902 together. One or more of the bridges 912 has a first end 914, a second end 916, and a multiple brace elements 918 therebetween. The first end 914 of the bridge 912 is coupled with the distal end 910 of a first ring 902 at a first connection point 920, and the second end 916 of the bridge 912 is coupled with the proximal end 908 of the adjacent second ring 902 at a second connection point 922. The first connection point 920 is circumferentially offset relative to the second connection point 922 so that the bridge 912 is transverse to the longitudinal axis. Each brace element 918 of one bridge 912 engages a brace element 918 of the adjacent bridge 912 when the corresponding adjacent rings 902 are in the contracted configuration, thereby providing additional support and rigidity to the stent 900 to lessen buckling of the stent during loading onto a delivery catheter or during deployment therefrom.

The plurality of interconnected struts 904 form a series of peaks and valleys in some examples. The peaks and valleys of a first ring 902 can be in phase with the peaks and valleys of an adjacent ring 902. The connector 906 that interconnects the plurality of struts 904 can be U-shaped or V-shaped. The rings 902 can be self-expanding, balloon expandable, or a combination thereof.

The one or more bridges 912 can comprise a first arm 924 and a second arm 926, and the brace element 918 disposed therebetween. The first arm 924 or the second arm 926 can comprise a linear strut. The first arm 924 or the second arm 926 can comprise a width, and the first brace element 918 can comprise a width wider than the width of the first arm 924 or the second arm 926. The first connection point 920 can be a peak of one ring 902, and the second connection point 922 can be on a valley of the adjacent ring 902. The first connection point 920 can be on the apex of the peak, and the second connection point 922 can be on the bottom of the valley.

A bridge 912 can couple each pair of adjacent struts 904 interconnected together in a first ring 902 with a pair of adjacent struts 904 interconnected together in an adjacent second ring 902 or an adjacent third ring 902. The first brace element 918 can comprise a rectangular region, and can also comprise an upper engagement surface and a lower engagement surface. The upper engagement surface can engage a lower engagement surface on an adjacent brace element 918 when the corresponding rings 902 are in the collapsed configuration. The upper and lower engagement surfaces can comprise flat planar surfaces. The upper engagement surface can have a first contour and the lower engagement surface on the adjacent brace element 918 can have a second contour, and the first contour can nest within the second contour.

The one or more bridges 912 can comprise a plurality of bridges 912 each having single or multiple brace elements 918. The bridges 912 can join the two adjacent rings 902 together, and the brace elements 918 on each bridge 912 can be axially aligned with one another to form a circumferentially oriented column of brace elements 918. The brace elements 918 on each bridge 912 can be circumferentially aligned with one another to form an axially oriented row of brace elements 918. The brace element 918 on a first bridge 912 can be axially offset relative to the brace element 918 on the adjacent ring 902, thereby forming a staggered pattern of brace elements 918. The brace elements 918 can also be arranged to form a circumferentially staggered pattern.

A first bridge 912 can couple a first ring 902 and a second adjacent ring 902, and a second bridge 912 can couple the second ring 902 with a third ring 902 adjacent the second ring 902. The first bridge 912 can have a first slope, and the second bridge 912 can have a second slope opposite the first bridge 912. The first brace element 918 may not contact a brace element 918 of an adjacent bridge 912 when the corresponding rings 902 are in the radially expanded configuration. The plurality of bridges 912 can be disposed between adjacent rings 902 and can be substantially parallel with one another.

One or more of the bridges 912 can comprise a length, and each brace element 918 comprises a length shorter than the length of the bridge 912. The one or more bridges 912 can comprise a second brace element 918 separated from the first bridge 912 by the strut 904. The one or more bridges 912 can comprise a plurality of bridges 912 each having the first brace element 918 and the second brace 918 separated by the strut 904. The plurality of bridges 912 can join two adjacent rings 902 together, and the first and second brace elements 918 on each bridge 912 can be circumferentially aligned with one another, thereby forming a first column of circumferentially oriented brace elements 918 and a second column of circumferentially oriented brace elements 918.

The first brace element 918 can comprise a slotted region extending through the entire thickness of the brace element 918. The first brace element 918 can comprise a solid tab without slots extending therethrough. A pair of bridges 912 each having the brace element 918 and joining two adjacent rings 902 can be separated by the bridge 912 without the brace element 918 and joining the two adjacent rings 902. At least some of the plurality of interconnected struts 904 can remain unconnected with a bridge 912. At least some of the bridges 912 can comprise the brace element 918 having a tapered proximal or distal end.

Thus, the stent 900 defines an extra-long braced stent in a radially collapsed configuration. That is, the bridges 912 including a plurality of brace elements 918 (three are shown merely for example) that are longer from the first end 914 to the second end 916 than the length of any of the rings 902.

In another aspect of various embodiments, a stent 1000 comprises a plurality of radially expandable rings 1002 each having a contracted configuration suitable for delivery and a radially expanded configuration for engaging and supporting tissue. The stent 1000 defines a helical braced stent in various embodiments as described below. Each ring 1002 is formed from a plurality of interconnected struts 1004, with adjacent struts 1004 in each ring 1002 being connected together with a connector 1006, and each ring 1002 having a proximal end 1008, and a distal end 1010. The plurality of rings 1002 are coaxially aligned with one another to form a longitudinal axis. The distal end 1010 of one ring 1002 faces the proximal end 1008 of the adjacent ring 1002. The connection point of the proximal ring 1002 is circumferentially offset relative to the distal ring 1002 so as to create a helical pattern along the length of the stent 1000.

The stent 1000 also has a plurality of extra-long bridges 1012 disposed between adjacent rings 1002. The plurality of the bridges 1012 couple adjacent rings 1002 together. One or more of the bridges 1012 can comprise a first end, a second end, and a multiple brace elements 1012 therebetween. The first end of the bridge 1012 is coupled with the distal end 1010 of the first ring 1002 at a first connection point, and the second end of the bridge 1012 is coupled with the proximal end 1008 of the adjacent second ring 1002 at a second connection point. The first connection point is circumferentially offset relative to the second connection point so that the bridge 1012 is transverse to the longitudinal axis. Each brace element 1014 of one bridge 1012 engages a brace element 1014 of the adjacent bridge 1012 when the corresponding adjacent rings 1002 are in the contracted configuration, thereby providing additional support and rigidity to the stent to lessen buckling of the stent during loading onto a delivery catheter or during deployment therefrom.

Variations and modifications are contemplated. For example, a stent can comprise a plurality of radially expandable rings each having a contracted configuration suitable for delivery and a radially expanded configuration for engaging and supporting tissue. Each ring is formed from a plurality of interconnected struts, with adjacent struts in each ring being connected together with a connector, and each ring having a proximal end, and a distal end. These connectors can be polymeric or metallic. The plurality of rings is coaxially aligned with one another to form a longitudinal axis. A distal end of one ring faces a proximal end of an adjacent ring. The stent also has a plurality of bridges disposed between adjacent rings. The plurality of the bridges couple adjacent rings together. One or more of the bridges comprise a first end and a second end. The first end of the bridge is coupled with the distal end of a first ring at a first connection point, and the second end of the bridge is coupled with the proximal end of an adjacent second ring at a second connection point. The first connection point is circumferentially offset relative to the second connection point so that the bridge is transverse to the longitudinal axis. Each brace element of one bridge engages a brace element of the adjacent bridge when the corresponding adjacent rings are in the contracted configuration thereby providing additional support and rigidity to the stent to lessen buckling of the stent during loading onto a delivery catheter or during deployment therefrom.

The stent described above and, in particular the polymeric connector, can be used to deliver drugs to tissue, support tissue, or maintain patency of body lumens. Antiplatelet agents, anticoagulant agents, antimicrobial agents, anti-inflammatory agents, and other drugs can be supplied in stents to reduce the incidence of restenosis or to maintain lumen patency.

Thus, various embodiments provide stent configurations that are more fracture resistant. For example, the stent configurations allow the stent, once positioned in the anatomy, to deform and return to an original shape, or move, as needed, to prevent fracturing. Additionally, the stents described herein can have portions formed from different types (e.g., braided and wireform) or having ends with different properties (e.g., different diameters or different numbers of stents, such as in the bifurcated design).

Any range or device value given herein may be extended or altered without losing the effect sought, as will be apparent to the skilled person.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f) paragraph, unless and until such claim limitations expressly use the phrase “means for” followed by a statement of function void of further structure.

What is claimed is:

1. A stent comprising:
at least one first stent portion formed from a first stent type; and
at least one second stent portion formed from a second stent type, the second stent portion coupled to the first stent portion, and the first stent type being different than the second stent type.

2. The stent of claim 1, wherein the first stent portion comprises a braided stent portion, the braided stent portion comprising overlapping interwoven filaments forming a tubular first tubular stent portion, and the second stent portion comprises a wireform stent portion coupled to an end of the braided stent portion.

3. The stent of claim 2, wherein the interwoven filaments comprise one or more radiopaque filaments.

4. The stent of claim 1, wherein the first stent portion comprises a braided stent portion and the second stent portion comprises ring stents, the ring stents positioned at ends of the braided stent portion within the braided stent portion.

5. The stent of claim 4, wherein the braided stent portion is formed from a shaped memory metal.

6. The stent of claim 1, wherein filaments forming at least one of the first stent portion or the second stent portion comprise longitudinal filaments that are radiopaque, metallic or polymeric.

7. The stent of claim 1, wherein filaments forming at least one of the first stent portion or the second stent portion are configured to deliver drugs.

8. The stent of claim 1, wherein the first stent portion comprises a main stent portion and the second stent portion comprises bifurcated stent portions split from the main stent portion.

9. The stent of claim 1, wherein at least one of the first stent portion or the second stent portion comprises a tapered diameter that changes along a length thereof.

10. The stent of claim 1, wherein the first stent portion comprises a radially expandable ring and the second stent portion comprises a plurality of bridges and braces having a length greater than a length of the radially expandable ring.

11. The stent of claim 1, wherein the first stent portion comprises a plurality of radially expandable rings and the second stent portion comprises a plurality of bridges, wherein a connection point of a proximal ring of the radially expandable rings is circumferentially offset relative to a distal ring to define a helical pattern along a length of the stent.

12. A stent comprising:
a plurality of filaments forming a tubular structure;
a first end of the tubular structure having a first property; and
a second end of the tubular structure having a second property, wherein the first property is different than the second property.

13. The stent of claim 12, wherein the first and second properties are first and second diameters, the first diameter being greater than the second diameter.

14. The stent of claim 12, wherein end portions of the plurality of filaments at the first and second ends are folded back to form smooth ends.

15. The stent of claim 12, wherein the plurality of filaments comprises overlapping interwoven filaments forming the tubular structure.

16. The stent of claim 15, wherein the overlapping comprises a repeating overlapping pattern.

17. The stent of claim 15, wherein the overlapping comprises a non-repeating overlapping pattern.

18. The stent of claim 12, wherein the first property comprises a main diameter and the second property comprises bifurcated diameters.

19. A stent comprising:
a plurality of rings, each of the rings comprising a plurality of struts; and
a plurality of bridges interconnecting at least some of the rings, the plurality of bridges comprising a plurality of braces, wherein the plurality of braces has a length greater than the length of each of the rings.

20. The stent of claim 19, wherein the rings are offset from each other to form a helical pattern along a length of the stent.
